(19) United States
(12) Patent Application Publication
(10) Pub. No.: US 2015/0371401 A1
(43) Pub. Date: Dec. 24, 2015

(54) Methods and Systems for Imaging Tissue Motion Using Optical Coherence Tomography

(71) Applicant: UNIVERSITY OF WASHINGTON, Seattle, WA (US)

(72) Inventors: Ruikang K. WANG, Seattle, WA (US); Murray JOHNSTONE, Bainbridge Island, WA (US)

(21) Appl. No.: 14/761,435

(22) PCT Filed: Mar. 13, 2014
(86) PCT No.: PCT/US2014/025853

§ 371 Date: Jul. 16, 2015

Related U.S. Application Data
(60) Provisional application No. 61/780,407, filed on Mar. 13, 2013.

(51) Int. Cl. G06T 7/20; G06K 9/62; A61B 5/00; A61B 3/00; A61B 3/12; G06T 7/00; A61B 3/10

(52) U.S. Cl. CPC G06T 7/20; G06T 7/0012; G06T 7/0081; G06K 9/6201; A61B 3/102; A61B 3/0025; A61B 3/1241; A61B 5/4836; A61B 5/4842; A61B 5/7282; G06T 2207/30041; G06T 2207/30104

(57) ABSTRACT

A system and method for measuring tissue motion within a living tissue of the fundus of the eye, including the ONH, in a subject are provided. A phase-sensitive OCT using time-lapse B-scans is provided to measure movement of fundus tissue and isolate the ONH component of the tissue, allowing for accurate evaluation of pulse-induced ONH movement. Phase information from retina tissue near the ONH may further be used as a reference to compensate for bulk tissue movement artifact. Furthermore, images of a central retinal artery or a central retinal vein pulse from the subject may be used to define and correlate a pulsatile blood flow with the ONH tissue movement for examination.

### CROSS-REFERENCE TO RELATED APPLICATIONS

This application claims priority to U.S. Provisional Patent Application Ser. No. 61/780,407 filed on Mar. 13, 2013, which is hereby incorporated by reference in its entirety.

### BACKGROUND

Optical coherence technology (OCT) is a non-contact, noninvasive, real-time imaging modality that is capable of cross-sectional imaging of biological tissue with high spatial resolution.

Currently, OCT is used to provide static, structural images of a biological sample in vivo. Movements of tissues or fluids within the biological sample are difficult to monitor and measure, however. Because of this difficulty, current understanding of organs such as the eye, which contains moving tissues such as the optic nerve head (ONH), fundus, choroid, retina, optic nerve layer, and ciliary body, for example, is limited. For example, current structural OCT techniques cannot distinguish axial motion of the entire globe of the eye from axial motion specific to the ONH.

This limited understanding of physiologically important tissue movement that is important to normal function can hinder diagnosis and successful treatment of any problems in the tissue. An ability to characterize and/or image tissue motion is also important for quantitative assessment of the tissue biomechanical properties, changes in biomechanical properties as a result of disease processes and subsequent diagnosis, prognosis, or treatment of any issues or functional abnormalities associated with the tissue.

There is a need for a noncontact method and system for visualization of movement within a biological sample.

### SUMMARY

In accordance with the present invention, a system and a method are defined for measuring tissue motion within in a living tissue of an eye in a subject.

In one embodiment, the method may comprise extracting tissue motion from a plurality of images acquired from the living tissue using an optical coherence tomography system. The extracting may comprise acquiring images of a region including at least a portion of an optical nerve head (ONH) tissue of the subject, defining phase differences between the images to extract tissue movement within the region, isolating ONH tissue movement from bulk tissue movement for the extracted tissue motion within the region, and mapping the isolated ONH tissue movement for examination.

The method may further comprise acquiring images of a central artery or a central retinal vein pulse from the subject, defining a pulsatile blood flow from the acquired images for a given time period, and correlating the ONH tissue movement and the pulsatile blood flow for examination. Correlating the ONH tissue movement and the pulsatile blood flow may include correlating time and phase differences between the ONH tissue movement and the pulsatile blood flow. The ONH tissue movement may additionally be normalized as a function of an amplitude, velocity or waveform of the pulsatile blood flow.

In another embodiment, a system for measuring tissue motion in a living tissue is provided. The system comprises an OCT probe, an optical circulator, coupler, a spectrometer, and a physical computer readable storage medium. The OCT probe, optical circulator, coupler, and spectrometer are used to acquire images of the living tissue. The physical computer readable storage medium comprises instructions executable to perform functions to extract tissue motion from the acquired images including acquiring images of a region including at least a portion of an optical nerve head (ONH) tissue of the subject, defining phase differences between the images to extract tissue movement within the region, isolating ONH tissue movement from bulk tissue movement for the extracted tissue motion within the region, and mapping the isolated ONH tissue movement for examination.

The system and method provide measurement of tissue motion in a living tissue, such as an ocular tissue,and may provide measurement of tissue motion the ONH. The measurement of tissue motion may include a measure me of and analyses of relationships between one or more of the following: pulsatile axial movements of any tissue of the ONH, fundus, choroid, retina, optic nerve fiber layer, and ciliary body; tissue velocity of movement and changes over time; amplitude of displacement of tissue and changes over time; waveforms of tissue motion and changes over time; waveforms of the central retinal artery and central retinal vein pulse and changes over time; comparative analyses between waveforms of tissue motion and waveforms of the central retinal artery and central retinal vein pulse and changes over time; phase and time differences between the central retinal artery and central retinal vein pulse motion and tissue motion and changes over time; harmonic analysis of the waveforms of tissue motion and changes over time; and evaluation of the ratio of the first harmonic strength to the second harmonic strength.

The system and method may be used to diagnose, provide a prognosis, monitor treatment, and guide treatment decisions for a disorder of the living tissues of the ONH.

The system and method may be used for a subject at risk of any ocular disorder, including but not limited to an ONH disorder. The ONH disorders may include any type of glaucoma, including but not limited to one or a combination of the following: open angle glaucoma, closed angle glaucoma, secondary glaucoma, pigmentary glaucoma, pseudoexfoliation glaucoma, uveitic glaucoma, neovascular glaucoma, low tension glaucoma and other glaucoma which either have a currently known or a currently unrecognized etiology. The system and method may be used to determine whether a subject is likely to respond to treatment of the ONH, monitor the efficacy of treatment of the ONH, make a treatment decision based on a prognosis related to use of the system and method to determine the functional status, guidance in medical, laser or surgicial intervential decisions based on system and device-dependent measurements that provide information about the functional status of the ONH and the likelihood of success of alternative interventions. Furthermore, the system and method may be used to determine the likely rate of progression of the disease associated with the ocular pathology.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a schematic of an exemplary system in accordance with at least one embodiment;

FIG. 2a depicts a fundus image of the ONH, generated from the system of FIG. 1, in accordance with at least one embodiment;

FIG. 2b depicts a structural cross-section image of the image depicted in FIG. 2a, in accordance with at least one embodiment;

FIG. 2c depicts a phase difference map corresponding to the structural cross-section image of FIG. 2b, in accordance with at least one embodiment;

FIG. 2d depicts a graph illustrating phase difference data between adjacent B-scans plotted over time, in accordance with at least one embodiment:

FIG. 2e depicts a graph illustrating tissue motion after compensating for bulk tissue motion corresponding to the phase difference data of FIG. 2d plotted over time, in accordance with at least one embodiment;

FIG. 2f depicts a graph illustrating phase difference data between adjacent B-scans plotted over time when there is phase wrapping, in accordance with at least one embodiment;

FIG. 2g depicts a graph illustrating the phase difference data of FIG. 2f after phase unwrapping, in accordance with at least one embodiment;

FIG. 3a depicts an exemplary velocity map of ONH movements over a time period, in accordance with at least one embodiment:

FIG. 3b depicts an exemplary velocity the position marked by the line of FIG. 3a, in accordance with at least one embodiment;

FIG. 3c depicts a frequency analysis of the velocity curve of FIG. 3b, corresponding to the downward velocity relative to the probe beam;

FIG. 3d depicts a displacement map of the ONH, corresponding to the upward velocity relative to the probe beam;

FIG. 3e depicts a displacement curve corresponding to the velocity curve of FIG. 3b, in accordance with at least one embodiment;

FIG. 3f depicts a frequency analysis of the displacement curve of FIG. 3e, in accordance with at least one embodiment;

FIG. 3g depicts a structural cross-sectional image of the ONH;

FIG. 3h depicts a corresponding blood flow map for FIG. 3g, in accordance with at least one embodiment;

FIG. 4a depicts dynamic blood flow measured from a central retinal artery of a subject;

FIG. 4b depicts frequency analysis of the dynamic blood flow of FIG. 4a; and

FIG. 5 depicts a simplified flow diagram of an example method that may be carried out to measure tissue motion within a living tissue, in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein, it will be readily understood that the aspects of the present disclosure, as generally described herein, and illustrated in the figures, can be arranged, substituted, combined, separated, and designed in a wide variety of different configurations, all of which are explicitly contemplated herein.

Abnormal biomechanical properties of the ONH may play important role in a number of eye diseases, including glaucoma. However, the extent to which ONH biomechanical properties determine susceptibility to damage from elevated intraocular pressure, for example, is unknown because functional measurement tools are lacking that are capable of measuring ONH movement.

A phase-sensitive OCT using time-lapse B-scans is provided to measure micron-scale movement of fundus tissue and isolate the ONH component of the tissue, allowing for accurate evaluation of pulse-induced ONH movement. Phase information from retina tissue near the ONH may be used, as discussed below, as a reference to compensate for bulk tissue movement artifact (due to gross patient movement).

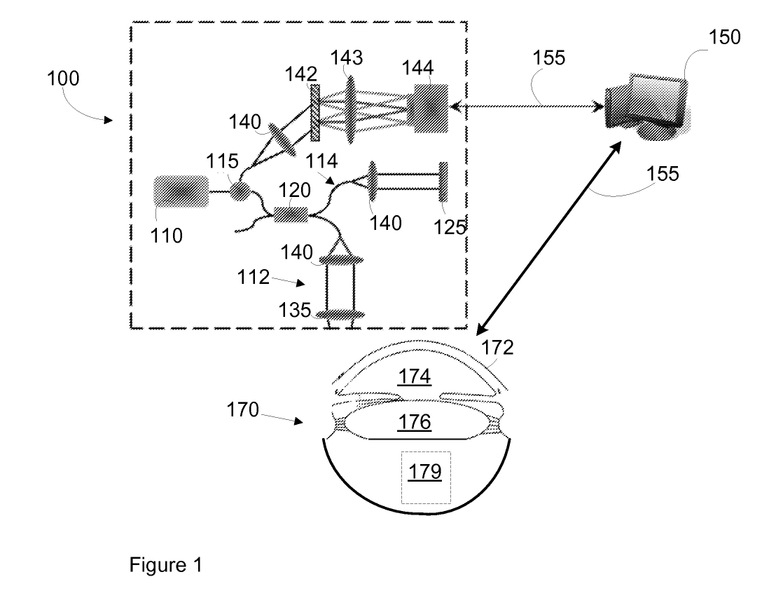

FIG. 1 depicts a schematic of an exemplary system 100 in accordance pith at least one embodiment. The system may be used, among other things, to measure tissue motion within a living tissue sample of a subject. Thus, the system 100 may be used on a subject in vivo. As referenced herein, a subject may be a human subject.

In FIG. 1, an OCT system is shown as system 100. The system 100 may include a light source 110, a nonreciprocal optical element 115, a fiber coupler 120, a reference mirror 125, an objective lens 135, a plurality of collimating lenses 140, a diffraction grating 142, a focusing lens 143, and one or more spectrometers 144. The system 100 may further include a computing system 150. A sample 170 to be imaged is also shown in FIG. 1. The OCT system may be a time-domain OCT system, spectral domain OCT (SD-OCT) system, swept source OCT system.

The OCT system may be a phase-sensitive OCT (PhS-OCT) system. In one example embodiment, the OCT system may be an OCT system based on a SD-OCT configuration.

In one example embodiment, the light source 110 may be a low temporally coherent light source, such as a broadband superluminescent diode. In other embodiments, other light sources may be used. In one example embodiment, the light source 110 has a central wavelength within the range of about 400--1850 nm. The light source may have a central wavelength of about 850 nm, for example. In another example embodiment, the light source may have a central wavelength of about 1050 nm. In yet another example embodiment the light source may have a central wavelength of about 1310 nm. In yet another example embodiment, the light source may have a central wavelength of about 1350 nm. In one example embodiment, the light source 110 has a spectral bandwidth of about 60 nm.

The nonreciprocal optical element 115 may be an optical circulator, and may have a first port connected to receive light from the light source 110. The nonreciprocal optical element 115 may further include a second port that may direct light from the first port to the fiber coupler 120 and receive light back from the fiber coupler 120, and a third port for directing light received from the fiber coupler 120 to the spectrometer 144.

The fiber coupler 120 serves as a beamsplitter, which transmits or splits some fraction of the power of the incident light power from the light source 110 into each of a sample arm 112 and a reference arm 114. Light returning from both the sample and the reference arms 112 and 114 may be fed to the one or more spectrometers 144 via the nonreciprocal optical element 115. In one example embodiment, the fiber coupler 120 may comprise a pair of fibers partially fused together. The fiber coupler may be a 2×2 fiber coupler.

The reference mirror 125 serves to reflect light directed from the fiber coupler 120 back to the fiber coupler 120.

The fiber coupler 120 feeds light to a collimating lens 140 of the sample arm 112, which is then focused by the objective lens 135 onto the sample 170. In one example embodiment, the objective lens 135 may comprise a focal length of about 50 mm.

The grating 142 may serve to split and diffract light into several light beams that travel in different directions.

The focusing lens 143 may serve to focus the light beams received from the grating 142 into the one or more spectrometers 144.

In one example embodiment, the one or more spectrometers 144 may comprise two spectrometer units, each comprising a camera, such as a charge coupled device (CCD) line scan camera. The CCD line scan camera may provide scanning at about a 500 kHz A-line scan rate with an axial resolution of about 7 μm in the air and 95 dB system sensitivity at an imaging depth of 0.5 nm. The sensitivity to the tissue movement may be as low as 0.3 nm, which is sufficient to measure small movements of the optic nerve head (ONH). With the 500 kHz A-line scan rate, the system 100 may achieve a frame rate of about 800 Hz.

The one or more spectrometers 144 may send their output to the computing system 150 for further processing.

The computing system 150 may include a processor, data storage, and logic. These elements may be coupled by a system or bus or other mechanism. The processor may include one or more general-purpose processors and/or dedicated processors, and may be configured to perform an analysis on the output from the spectrometer 144. An output interface may be configured to transmit output from the computing system to a display. The computing system 150 may be further configured to send trigger signals 155 to the one or more spectrometers 144. Trigger signals 155 may be sent by the computing system 150 to synchronize the spectrometers 144 when more than one spectrometer 144 is present in the system.

In operation, a subject is positioned at a designated location to allow for observation of desired biological tissues of the sample 170. In some example embodiments, the subject may position his or her head on a slit-lamp headrest to minimize bulk tissue movement due to head movement. In the example shown in the system 100, the sample 170 is an eye of a subject. A subject may further focus his or her eye on a fixation target to minimize eye movement artifact. The sample 170 comprises a cornea 172, an iris 174, a lens 176, and a region for examination 179 that includes the ONH. The sample 170 is observed in vivo in the example depicted in FIG. 1.

The light source 110 is directed through the nonreciprocal optical element 115 to the fiber coupler 120 which splits the light into the two arms 112 and 114, the reference arm 114 being directed at the reference mirror 125 and the sample arm 112 indicating the OCT probe beam being directed at the sample 170.

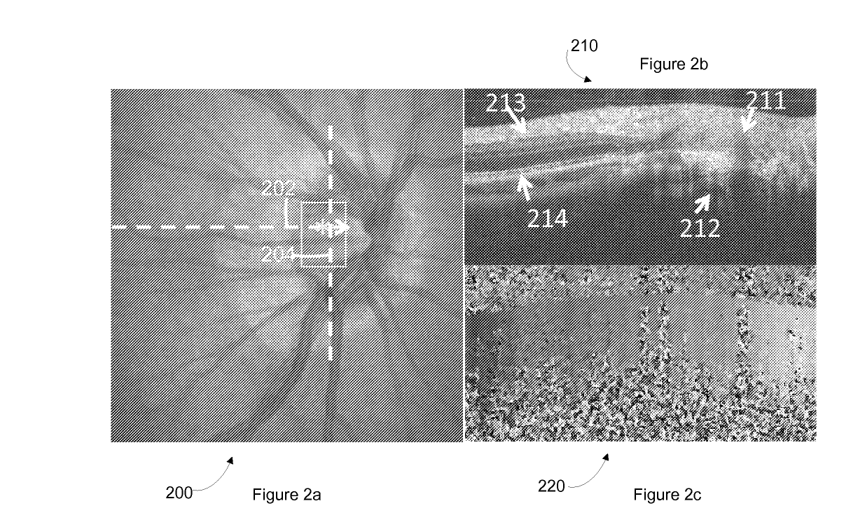

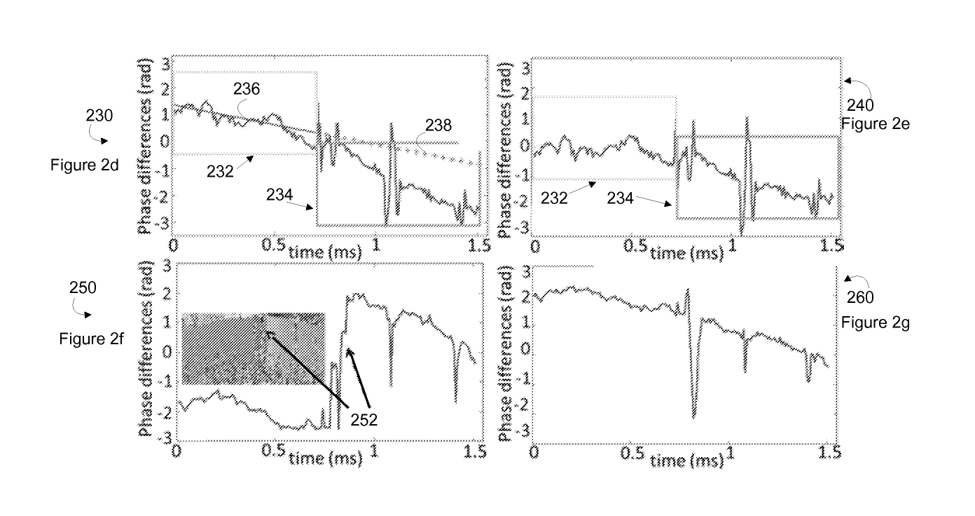

Light backscattered from the sample 170 in the sample arm 112 is then directed to the fiber coupler 120 and the nonreciprocal optical element 115, along with the reflected light from the reference mirror 125, which is then split via the grating and the various beams of light are then sent to the one or more spectrometers 144. The spectrometers 144 may then feed the output to the computing system 150 for further processing, as will be described with reference to FIGS. 2a-4b.

Before imaging ONH movements, such as ONH pulsatile movements, for example, an image using a traditional 4×4 mm² 3D scan of the ONH region may be taken. FIG. 2a depicts a fundus image 200 of the ONH, generated from a system such as the system 100 of FIG. 1, in accordance with at least one embodiment. Scanning positions are marked on the image 200 that indicate a scanning location used to measure pulsatile ONH movements 202, wherein an arrow indicates the scanning direction, and a scanning location to measure blood flow in the central retinal artery (CRA) 204.

In one example embodiment, for the sample 170 where the sample is a subject's eye, 600 A-lines may be captured to form one B-scan that covers about 3 mm in length. The scanning region may include both the ONH and the peripapillary retina to ensure extraction of the ONH motion.

Although the system 100 may achieve a frame rate of about 800 Hz as discussed above, other components, such as a galvanometer, may limit the frame rate. For example, if the galvanometer is only capable of maintaining a linear range of about 70% when working in a high speed mode, a more realistic frame rate may be about 500 Hz, allowing for a maximum detectable velocity out phase wrapping of about 105 μm/s.

In all, about 2600 repeated OCT B-frames may be captured at one spatial location for each dataset within about 5.2 seconds to provide coverage of about 5 human heart pulse cycles.

FIG. 2b depicts an example structural cross-section image 210 of the fundus image 200 depicted in FIG. 2a, in accordance with at least one embodiment. Various anatomic features are visible in the image 210: a pre-laminar layer 211, a laminar cribrosa 212, a retina 213, and a choroid 214.

After a repeated B-scan dataset has been acquired such as that described above, a phase difference map may be created between adjacent B-frames. FIG. 2c depicts a phase difference map 220 corresponding to the structural cross-section image 210 of FIG. 2b, in accordance with at least one embodiment. Tissue motion (ΔΦ) generated by both bulk tissue motion (ΔΦₘ) and ONH motion (ΔΦₒ) may be characterized as:

ΔΦ=ΔΦₘ+ΔΦₒ Equation 1

A histogram method may be performed on each A-line scan to obtain ΔΦ from the phase difference map.

The tissue motion ΔΦ may be presented as the following:

$$Δ Φ = \frac{4 n π v Δ t}{λ} + {ΔΦ}_{o} \qquad Equation 2$$

where n is the refractive index of the sample, v is the velocity of the tissue motion, λ is the central wavelength of the OCT system (e.g., 842 nm), and Δt is the time interval between adjacent B-frames (e.g., 2 ms). For in vivo imaging of a tissue that is in constant motion (including both bulk and localized motion), the tissue velocity v is a function of time.

The slit-lamp headrest securing a subject's head during the imaging process may minimize bulk tissue motion, providing for the bulk tissue motion to be a slow varying function of time, and allowing for the acceleration of bulk tissue motion to be considered constant within the short period of 2 ms between adjacent B-scans. Thus, the bulk tissue movement can be described by a first order polynomial function:

v=at+vᵢ Equation 3

where a is the acceleration and vᵢ is the initial velocity at the beginning of a B-scan.

The change in phase ΔΦ due to the tissue motion is also a function of time and can be represented as follows:

$$Δ Φ = \frac{4 n π a t Δ t}{λ} + Δ {Φ}_{o} + {Φ}_{i} \qquad Equation 4$$

where Φᵢ is due to the initial tissue velocity at the beginning of the B-scan.

FIG. 2d depicts a graph 230 illustrating phase difference data ΔΦ (in radians) between adjacent B-scans plotted over time, in accordance with at least one embodiment. Because of bulk motion of the eye, the ΔΦ is continuously decreasing within one B-scan, demonstrating that the acceleration of the bulk movements is almost constant.

As shown in graph 230, the ΔΦ curve may be partitioned into two regions: the peripapillary retina (retinal region 232) and the ONH (ONH region 234). The partitioning may be performed with the help and comparison with the OCT structural image, such as the image 220 of FIG. 2c.

Motion within the retinal region 232 is considered to result from bulk tissue movement, allowing for motion occurring in the retinal region 232 to be used as a reference to extract the movement in the ONH region 234.

A first order polynomial function may be fitted to the ΔΦ values in the retinal region (line 236) to determine the acceleration of the bulk tissue movement. Therefore, with known scan timing, the bulk tissue movement within the ONH region 234 can be extrapolated from this polynomial function, and is shown as broken line 238. The ΔΦ, due to the ONH movement can then be extracted by subtracting the extrapolated bulk tissue movement from the ΔΦ within the ONH region 234.

FIG. 2e depicts a graph 240 illustrating tissue motion after compensating for bulk tissue movement corresponding to the phase difference data (in radians) of FIG. 2d plotted over time, in accordance with at least one embodiment. As shown in graph 240, the values within the reference region 232 remain close to zero, whereas within the ONH region 234 the values deviate from the reference, demonstrating how ONH movement separates from bulk tissue movement.

For in vivo human imaging,the ΔΦ generated by tissue motion can sometimes be relatively large compared to the wavelength used. Under such conditions, the evaluated Δ and Φ would be phase wrapped because of the 2π-modulo in the sinusoidal function.

FIG. 2f depicts a graph 250 illustrating phase difference data between adjacent B-scans plotted over time when there is phase wrapping, in accordance with at least one embodiment. In the graph 250, the original ΔΦ is evaluated from each A-line. Because of phase wrapping, there is an abrupt step jump 252, shown in both an embedded image within the graph 250 and in the corresponding phase difference data, which may be corrected before proceeding to extract ONH movement.

To un-wrap the ΔΦ curve, a phase-unwrapping algorithm known in the art may be applied. Results from applying such an algorithm are shown in FIG. 2g. FIG. 2g depicts a graph 260 illustrating the phase difference data of FIG. 2f after phase-unwrapping, in accordance with at least one embodiment.

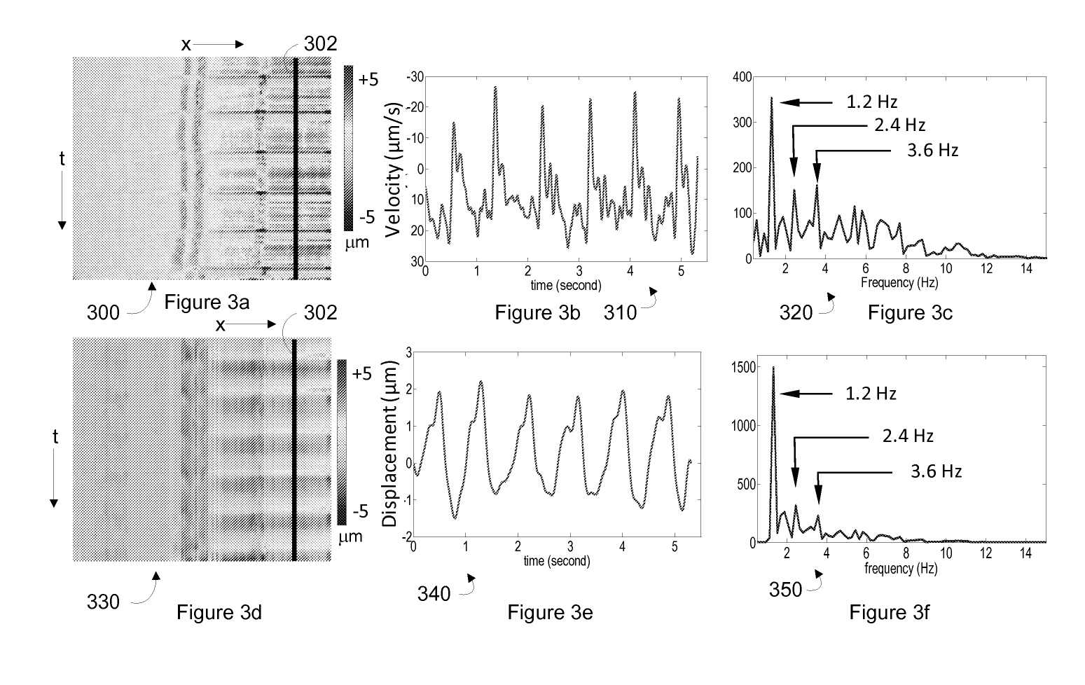

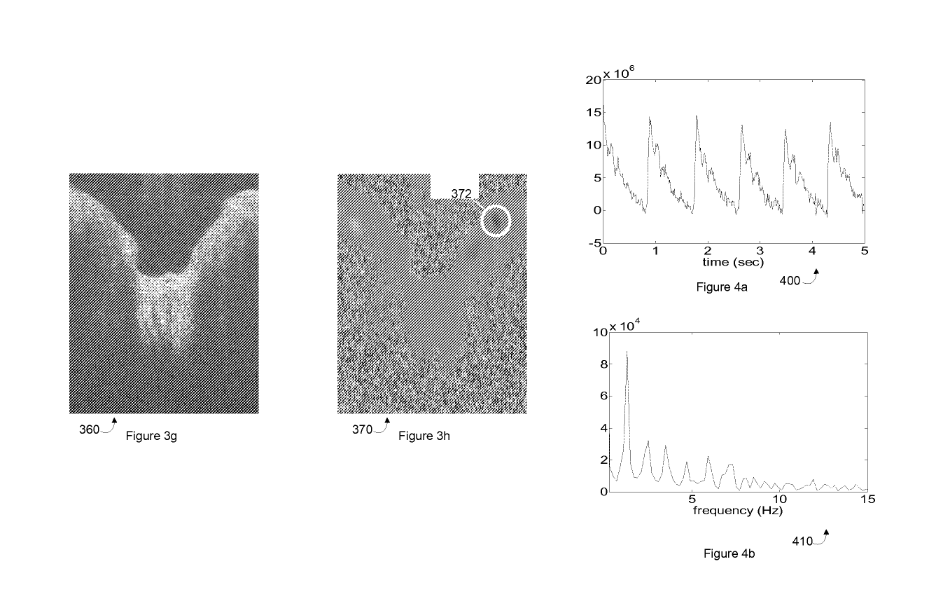

After ΔΦ curves are evaluated from the 2600 B-frames using the algorithms described above, the results may be stacked to produce a 2D motion or velocity map. FIG. 3a depicts an exemplary velocity map 300 of ONH movements over a time period, in accordance with at least one embodiment. In the velocity map 300, the horizontal axis represents scanning positions that correspond to the tissue position within the B-scan dataset, and the vertical axis represents the time lapse during the repeated B-scans. Oscillatory patterns caused by pulse-induced ONH movements are visible. A line 302 is marked for further analysis that is depicted and described with reference to FIG. 3b.

FIG. 3b depicts an exemplary velocity curve 310 at the position marked by the line 302 of FIG. 3a, in accordance with at least one embodiment. In FIG. 3b, the vertical axis depicts a magnitude ranging from −30 to 30 μm/sec.

FIG. 3c depicts a frequency analysis graph 320 of the velocity curve 310 of FIG. 3b, corresponding to the downward velocity relative to the probe beam. Graph 320 shows the frequency components of FIG. 3b after Fourier analyses. In addition to the fundamental frequency component of about 1.2 Hz, which may be approximately equivalent to a heartbeat of a subject, higher order harmonics (e.g. 2^{nd} and 3^{rd} harmonics) are also present as is known in the Fourier analyses of a real time-domain signal.

FIG. 3d depicts a displacement map 330 of the ONH, corresponding to the upward velocity relative to the probe beam. The displacement map 330 indicates displacement of the ONH tissue obtained through integrating FIG. 3a over the time t.

FIG. 3e depicts a displacement curve 340 corresponding to the velocity curve of FIG. 3b, in accordance with at least one embodiment. The displacement curve 340 depicts the displacement of the ONH extracted from the same position 302 as in FIG. 3b.

FIG. 3f depicts a frequency analysis graph 350 of the displacement curve 340 of FIG. 3e, in accordance with at least one embodiment. The frequency analysis graph 350 demonstrates both the fundamental and higher order harmonics in the signal.

FIG. 3g depicts a structural cross-sectional image 360 of the ONH, and FIG. 3h depicts a corresponding blood flow map 370 for FIG. 3g, in accordance with at least one embodiment. The image 360 is obtained by calculating the phase differences between adjacent A-lines. To evaluate pulsatile flow, the phase difference values of the CRA (circle 372 in FIG. 3h) may be integrated. The results from such an integration are shown in the graph 400 FIG. 4a, which depicts dynamic blood flow measured from the central retinal artery. The blood flow within the CRA is pulsatile. A comparison of the graphs of FIG. 3h and FIG. 3i reveals that the two curves are similar: they each have a steep increase at the beginning of each cycle followed by a slow decay after the cycle peak.

In an alternative embodiment, the pulsatile flow within the central retinal vein (CRV) may be used to perform the same or a similar evaluation.

FIG. 4b depicts frequency analysis graph 410 of the dynamic blood flow of FIG. 4a. The Fourier frequency analyses determined the fundamental frequency to be 1.2 Hz, equivalent to the heartbeat of the subject. CRA pulse frequency correlated with the fundamental frequency found in the ONH tissue movement.

Thus, correlating ONH tissue movement with a pulsatile blood flow, such as that of the CRA and/or CRV, may be performed. Comparative analyses between waveforms, as well as phase and time differences of tissue motion and waveforms of the CRA and/or the CRV pulse may thus be calculated and provided.

All of the above-described calculations may be performed by a computing system such as the computing system 150. Statistical analysis software may be present on the computing system to perform the various calculations.

The sample 170, as described above, may be a living ocular tissue, specifically in the ONH. In one example embodiment, the sample 170 may be the ONH of the eye and may provide measurement of tissue motion within the ONH of the eye. The measurement of tissue motion may include: pulsatile axial movements of any tissue of the ONH, fundus, choroid, retina, optic nerve fiber layer, and ciliary body: tissue velocity of movement and changes over time; amplitude of displacement of tissue and changes over time; waveforms of tissue motion and changes over time; waveforms of the central retinal artery and central retinal vein pulse and changes over time; comparative analyses between waveforms of tissue motion and waveforms of the central retinal artery and central retinal vein pulse and changes over time; phase and time differences between the central retinal artery and central retinal vein pulse motion and tissue motion and changes over time; harmonic analysis of the waveforms of tissue motion and changes over time; and evaluation of the ratio of the first harmonic strength to the second harmonic strength.

The measurement of issue motion may be used to diagnose, provide a prognosis, monitor treatment and guide treatment decisions for a disorder of the sample 170 of a subject. The treatment may include medical, laser, or surgical intervention. In one example embodiment, the measurement of tissue motion may determine whether the subject is at risk of an ONH disorder or has ocular pathology that will result in that disorder, as well as providing a prognosis for likelihood of the subject to respond to treatment for the ocular pathology or monitoring the efficacy of treatment of the subject. The ocular pathology may comprise but is not limited to, for example any one or a combination of the following: open angle glaucoma, closed angle glaucoma, secondary glaucoma, pigmentary glaucoma, pseudoexfoliation glaucoma, uveitic glaucoma, neovascular glaucoma, low tension glaucoma and other glaucoma which either have a currently known or a currently unrecognized etiology.

A treatment decision may be based on the prognosis, monitoring or assessment of current properties of the entire ONH tissue region conducted in accordance with the measurement calculated with reference to FIG. 1. For example, a treatment may be based on the global or regional behaviors or properties of the tissues. Behaviors of the tissues may include tissue motion as measured in accordance with the system and method of FIG. 1.

Thus, the velocity pulse of the central retinal may be captured using the OCT optimized for imaging of the retina and choroid, followed by a phase compensation algorithm that removes bulk motion allows for the quantitative characterization of the pulsatile tissue motion.

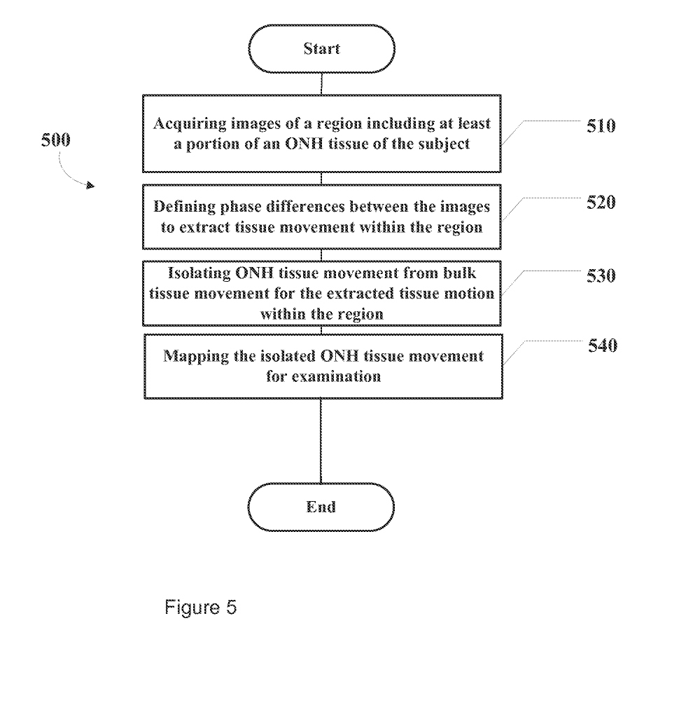

FIG. 5 depicts a simplified flow diagram of example method that may he carried out to measure tissue motion within a living tissue, in accordance with at least one embodiment. Method 500 shown in FIG. 5 presents an embodiment of a method that, for example, could be used with the system 100.

In addition, for the method 500 and other processes and methods disclosed herein, the flowchart shows functionality and operation of one possible implementation of the present embodiments. In this regard, each block may represent a module, a segment, or a portion of program code, which includes one or more instructions executable by a processor for implementing specific logical functions or steps in the process. The program code may be stored on any type of computer readable medium, for example, such as a storage device including a disk or hard drive. The computer readable medium may include a physical and/or non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. Alternatively, program code, instructions, and/or data structures may be transmitted via a Communications network via a propagated signal on a propagation medium (e.g., electromagnetic wave(s), sound wave(s), etc.).

The method 500 allows for extracting tissue motion from a plurality of images acquired from the living tissue using an OCT system. The OCT system may be the same or similar to the system 100 of FIG. 1. The method 500 may be used to diagnose, develop a prognosis, or monitor treatment for a disorder of the living tissue.

Initially, the method 500 includes acquiring images of a region including at least a portion of an ONH tissue of the subject, at block 510.

The method 500 then includes defining phase differences between the images to extract issue movement within the region, at block 520.

The method 500 includes isolating ONH tissue movement from bulk tissue movement for the extracted tissue motion within the region, at block 530.

The method 500 includes mapping the isolated ONH tissue movement for examination, at block 540.

The computing system 150 may plot the results, as described with reference to FIGS. 2d-3f.

While various aspects and embodiments have been disclosed herein, other aspects and embodiments will be apparent to those skilled in the art. The various aspects and embodiment; disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

1. A method of measuring tissue motion within a living tissue of an eye in a subject comprising:
extracting tissue motion from a plurality of images acquired from the living tissue using an optical coherence tomography system, wherein the extracting comprises:
acquiring images of a region including at least a portion of an optical nerve head (ONH) tissue of the subject;
defining phase differences between the images to extract tissue movement within the region;
isolating ONH tissue movement from bulk tissue movement for the extracted tissue motion within the region; and
mapping the isolated ONH tissue movement for examination.

2. The method of claim 1, further comprising:
acquiring images of a central retinal artery or a central retinal vein pulse from the subject;
defining a pulsatile blood flow from the acquired images for a given time period; and
correlating the ONH tissue movement and the pulsatile blood flow for any of comparison and normalization.

3. The method of claim 2, wherein correlating the ONH tissue movement and the pulsatile blood flow includes correlating time and phase differences between the ONH tissue movement and the pulsatile blood flow.

4. The method of claim 2, further comprising:
normalizing ONH tissue movement as a function of an amplitude of the pulsatile blood flow.

5. The method of claim 1, wherein measuring tissue motion within the living tissue of the eye comprises measuring one or more of the following, including analyses of relationships between one or more of the following: pulsatile axial movements of any tissue of the ONH, fundus, choroid, retina, optic nerve fiber layer, and ciliary body; tissue velocity of movement and changes over time; amplitude of displacement of tissue and changes over time; waveforms of tissue motion and changes over time; waveforms of the central retinal artery and central retinal vein pulse and changes over time; comparative analyses between waveforms of tissue motion and waveforms of the central retinal artery and central retinal vein pulse and changes over time; phase and time differences between the central retinal artery and central retinal vein pulse motion and tissue motion and changes over time; harmonic analysis of the waveforms of tissue motion and changes over time; evaluation of the ratio of the first harmonic strength to the second harmonic strength.

6. The method of claim 1, wherein the method is used to diagnose, provide a prognosis, monitor treatment, or provide guidance in medical, laser or surgical management for a disorder of the living tissue of the eye.

7. The method of claim 1, wherein the subject is at risk of an ocular pathology or has an ocular pathology.

8. The method of claim 7 wherein the ocular pathology is glaucoma.

9. The method of claim 7, wherein the subject is at risk of an ocular pathology and the method comprises diagnosing whether the subject has an ocular pathology.

10. The method of claim 7, wherein the subject has an ocular pathology and the method comprises determining the likely rate of progression associated with the ocular pathology.

11. The method of claim 7, wherein the subject has an ocular pathology and the method comprises providing a prognosis based on the extracted ONH tissue movement for whether the subject is likely to respond to treatment for the ocular pathology.

12. The method of claim 7, wherein the subject has an ocular pathology and the method comprises monitoring efficacy of treatment by monitoring the extracted ONH tissue movement of the subject for the ocular pathology.

13. The method of claim 11, further comprising making a treatment decision based on the prognosis or the monitoring.

14. The method of claim 10, further comprising making a treatment decision based on the measured tissue motion.

15. (canceled)

16. The method of claim 1, further comprising:
deriving biomechanical information concerning the living tissue from the isolated ONH tissue movement.

17. The method of claim 2, wherein acquiring the images of the central retinal artery pulse is simultaneous with acquiring images of the ONH tissue movement.

18. The method of claim 1, wherein the images are acquired using the optical coherence tomography system by a method comprising:
applying light from a low coherence light source with a central wavelength of about 400-1850 nm through an optical coupler that splits light from the light source to the living tissue and to a mirror;
recombining light reflected from the living tissue and the mirror through the optical coupler; and
sending the recombined reflected light through a grating to a spectrometer.

19. A system for measuring tissue motion within a living tissue comprising:
an optical coherence tomography probe;
an optical circulator;
a coupler;
a spectrometer;
a digital pulsimeter; and
a physical computer-readable storage medium;
wherein the system acquires images from the living tissue,
wherein the physical computer-readable storage medium has stored thereon instructions executable by a device to cause the device to perform functions to extract tissue motion from the acquired images, the functions comprising:
extracting tissue motion from a plurality of images acquired from the living tissue using an optical coherence tomography system, wherein the extracting comprises:
acquiring images of a region including at least a portion of an optical nerve head (ONH) of the subject;
defining phase differences between images to extract tissue movement within the region;
isolating ONH tissue movement from bulk tissue movement for the extracted tissue movement within the region; and
mapping the isolated ONH tissue movement for examination.

20. The system of claim 19, wherein the system for measuring tissue motion comprises measuring one or more of the following, including analyses of relationships between one or more of the following: pulsatile axial movements of any tissue of the ONH, fundus, choroid, retina, optic nerve fiber layer, and ciliary body; tissue velocity of movement and changes over time; amplitude of displacement of tissue and changes over time; waveforms of tissue motion and changes over time; waveforms of the central retinal artery and central retinal vein pulse and changes over time; comparative analyses between waveforms of tissue motion and waveforms of the central retinal artery and central retinal vein pulse and changes over time; phase and time differences between the central retinal artery and central retinal vein pulse motion and tissue motion and changes over time; harmonic analysis of the waveforms of tissue motion and changes over time; evaluation of the ratio of the first harmonic strength to the second harmonic strength.
